(19) United States
(12) Patent Application Publication
(10) Pub. No.: US 2023/0101242 A1
(43) Pub. Date: Mar. 30, 2023

(54) SHEATH AND DELIVERY SYSTEMS AND METHODS FOR TRANSCATHETER DEVICE IMPLANTATION

(71) Applicant: Silk Road Medical, Inc., Sunnyvale, CA (US)

(72) Inventors: Brad Steele, Sunnyvale, CA (US); William Whealon, Sunnyvale, CA (US)

(21) Appl. No.: 17/951,727

(22) Filed: Sep. 23, 2022

Related U.S. Application Data
(60) Provisional application No. 63/248,091, filed on Sep. 24, 2021.

(51) Int. Cl. A61F 2/24

(52) U.S. Cl. CPC A61F 2/2466; A61F 2250/0098

(57) ABSTRACT

Arterial access sheaths for delivering a treatment, such as a transcatheter aortic valve treatment, having markers configured to illustrate orientation of the distal end region of the sheath. Related systems, devices, and methods are provided.

### CROSS-REFERENCE TO RELATED APPLICATIONS

This application claims the benefit of priority under 35 U.S.C. § 119(e) to Provisional Patent Application Ser. No. 63/248,091, filed Sep. 24, 2021. The disclosure of the provisional application is incorporated by reference in its entirety.

### BACKGROUND

The present disclosure relates to devices and methods for accurately deploying devices in a desired orientation relative to the anatomy, such as prosthetic heart valves.

Patients with defective aortic heart valves are often candidates for a replacement heart valve procedure. The conventional treatment is the surgical replacement of the heart valve with a prosthetic valve. This surgery involves a gross thorocotomy or median sternotomy, cardiopulmonary bypass and cardiac arrest, surgical access and excision of the diseased heart valve, and replacement of the heart valve with a prosthetic mechanical or tissue valve. Valves implanted in this manner have historically provided good long term outcomes for these patients, with durability of up to ten or fifteen years for tissue valves, and even longer for mechanical valves. However, heart valve replacement surgery is highly invasive, can require lengthy recovery time, and is associated with short and long term complications. For high surgical risk or inoperable patients, this procedure may not be an option.

Recently, a minimally invasive approach to heart valve replacement has been developed. This approach, known as transcatheter aortic valve implantation (TAVI) or replacement (TAVR), relies on the development of a collapsible prosthetic valve which is mounted onto a catheter-based delivery system. This type of prosthesis can be inserted into the patient through a relatively small incision or vascular access site, and may be implanted on the beating heart without cardiac arrest. The advantages of this approach include less surgical trauma, faster recovery time, and lower complication rates. For high surgical risk or inoperable patients, this approach offers a good alternative to conventional surgery. Examples of this technology are the Sapien Transcatheter Valve (Edwards Lifesciences, Irvine, Calif.) and the CoreValve System (Medtronic, Minneapolis, Minn.). U.S. Pat. No. 6,454,799, which is incorporated herein by reference in its entirety, describes examples of this technology.

There are two main pathways for valves inserted using the TAVI approach. The first is a vascular approach via the femoral artery (referred to as a transfemoral approach), either percutaneously or through a surgical cut-down and arteriotomy of the femoral artery. Once placed into the femoral artery, the valve mounted on the delivery system is advanced in a retrograde manner (in the reverse direction as blood flow) up the descending aorta, around the aortic arch, and across the ascending aorta in order to be positioned across the native aortic valve. Transfemoral aortic valve delivery systems are typically over 90 cm in length and require the ability to navigate around the aortic arch. The relatively small diameter of the femoral artery and the frequent presence of atherosclerotic disease in the iliofemoral anatomy limits the maximum diameter of the delivery system to about 24 French (0.312″) in diameter. The second pathway, termed transapical, involves accessing the left ventricle through the apex of the heart via a mini-thorocotomy, and advancing the valve delivery system in an antegrade fashion (in the same direction as blood flow) to the aortic valve position. This pathway is much shorter and straighter than the transfemoral path, but involves a surgical puncture and subsequent closure of the wall of the heart.

Other approaches have been described, including access from the subclavian artery, and direct puncture of the ascending aorta via a mini-thorocotomy. The subclavian approach (transsubclavian approach) has been used when the transfemoral route is contra-indicated, but may block flow to the cerebral vessel through the ipsilateral common carotid artery. A direct aortic puncture is usually considered if all other routes must be excluded due to anatomic difficulties including vascular disease. Puncture of the aortic wall, and subsequent closure, carries associated surgical risk including aortic dissection and rupture.

The transfemoral approach to the aortic valve, as opposed to the transapical or other alternative approaches, is a generally more familiar one to the medical community. Accessing the ascending aorta from the femoral artery is standard procedure for interventional cardiologists. Balloon valvuloplasty procedures via the transfemoral approach have been performed for years. The surgical approaches such as the transapical access or direct aortic puncture are less familiar and require practitioners with both surgical and endovascular skills; techniques for the surgical approaches are still evolving and whether they offer advantages over the transfemoral and transsubclavian methods have yet to be determined. However, problems also exist with the transfemoral and transsubclavian approaches. One is that the desired access vessel is often too small and/or is burdened with atherosclerotic disease, which precludes the artery as an access point. A second problem is that the pathway from the access point to the aortic valve usually involves one or more major turns of at least 90° with a relatively tight radii of curvature, 0.5″ or less, requiring a certain degree of flexibility in the delivery system. This flexibility requirement restricts the design parameters of both the valve and the delivery system, and together with the required length of the delivery system reduces the level of control in accurately positioning the valve.

TAVR also requires accurate placement of the new valve either into the diseased native valve, or into a previously implanted valve. Longitudinal and circumferential alignment of the prosthetic valve relative to the native anatomy is critical to achieve procedural success. Correct placement for apposition and not interfering with blood flow to the coronary arteries, in particular, are necessary. Clinically, the alignment of the implanted valve posts and leaflets relative to the native or implanted aortic valve commissures is referred to as commissural alignment. The long endovascular tracking length, particularly with transfemoral approach, significant curvature in the tracking pathway, as well as risk of vessel interruption due to damage and release of embolic particles during tracking are particular challenges of current TAVR implants and delivery systems that limit the ability for accurate valve placement.

Transfemoral approaches are limited by long endovascular tracking length and the significant curvatures through iliac and aortic arch. The risk of vessel interruption such as vessel damage and/or embolic particles breaking free during tracking is high. These limitations impact the ability of TAVR systems to provide accurate placement.

Typical TAVR procedures also involve the creation of multiple access sites. TAVR devices typically require a large bore femoral access sheath whereas diagnostic catheters are inserted through small bore femoral access. Small bore femoral vein access is used for inserting electrophysiology pacing leads. Small bore radial artery access may also be used for embolic protection device delivery. Multiple access sites used during the procedure limits success of TAVR procedures and involves additional patient risk, for example, due to the need to close multiple access sites and an increased risk in devices inadvertently contacting and moving other devices, such as a femoral artery delivered TAVR delivery system contacting a radial artery delivered embolic protection device.

### SUMMARY

In an aspect, disposed is an arterial access sheath for delivering a transcatheter aortic valve treatment including an elongate body having at least one internal lumen extending from a proximal opening to a distal opening; a proximal hub coupled to the proximal opening of the elongate body and configured to remain external to a patient, the proximal hub comprising one or more proximal orientation markings; and a radiopaque marker band embedded within a distal end region of the elongate body, the radiopaque marker band comprising cut-outs separated from one another around a circumference of the distal end region corresponding circumferentially to the one or more proximal orientation markings. The cuts-outs form gaps in radiopacity in the marker band creating at least two non-radiopaque distal orientation markings. Overlap of the at least two distal orientation markings indicates circumferential orientation of the distal end region of the elongate body.

The elongate body can have a length adapted to be introduced into an access site at a left or a right common carotid artery, or a left or a right subclavian artery. A shape of the distal orientation markings can form a second, different shape upon at least partial overlap of the at least two distal orientation markings with one another. The shape can be a rectangle, circle, triangle, diamond, arrow, hemisphere, or square. The at least two distal orientation markings can include at least two groups of distal orientation markings. The at least two groups can be distinguishable from each other. The at least two groups can be distinguishable by a shape of markers. The at least two groups can be distinguishable by a number of markers. The radiopaque marker band can include a polymer loaded with one or more radiopaque materials. The radiopaque materials can be tungsten, tantalum, barium sulfate, platinum, stainless steel, or gold. The distal orientation markings can additionally include a button of radiopaque material projecting away from a sidewall of the distal end region of the elongate body. The proximal orientation markings can be on a proximal-facing surface of the hub and can be directly visible by a user. The proximal orientation markings can include adhesive markings, painted markings, molded markings, etched markings, embossed markings, or printed marking on the proximal hub. The elongate body can have an asymmetry around its circumference, wherein the proximal orientation markings correspond to the asymmetry. The elongate body can be non-circular in cross-section.

In an interrelated aspect, provided is a method of treating an aortic valve including forming a penetration through the neck of a patient in a wall of a common carotid artery and introducing an arterial access sheath through the penetration. The access sheath includes an elongate body coupled to a proximal hub, the proximal hub configured to remain external to a patient and comprising one or more proximal orientation markings. The distal end region of the elongate body has a radiopaque marker band having cut-outs separated from one another around a circumference of the distal end region so as to correspond circumferentially to the one or more proximal orientation markings. The cut-outs form gaps in radiopacity in the marker band creating at least two non-radiopaque distal orientation markings. The method further includes aligning the distal end region of the elongate body relative to a target anatomy under fluoroscopy according to an overlapping circumferential position of the at least two distal orientation markings; introducing a prosthetic valve through the access sheath using as a guide the one or more proximal orientation markings corresponding to the at least two distal orientation markings; and deploying the prosthetic valve at or near the position of a native aortic valve.

In an interrelated aspect, provided is an arterial access sheath for delivering a transcatheter aortic valve treatment. The sheath includes an elongate body having a plurality of internal lumens extending parallel to one another between respective proximal openings and respective distal openings, the distal openings located at a distal-most end of the elongate body. The sheath includes an embolic protection device lumen extending between a proximal opening and an exit port, the exit port located through a sidewall of the elongate body a distance proximal to the distal-most end of the elongate body; a first radiopaque marker band embedded within a distal end region of the elongate body; and a second radiopaque marker band embedded within a region of the elongate body near the exit port. The sheath includes a proximal hub configured to remain external to a patient, the proximal hub coupled to the respective proximal openings of the elongate body and the proximal opening of the embolic protection device lumen.

The plurality of internal lumens can include an interventional device lumen having an inner diameter that is between 12 French and 24 French or between 4 mm and 8 mm. The plurality of internal lumens can include at least two auxiliary lumens having an inner diameter that is between 3 French and 9 French or between 1 mm and 3 mm. The sheath can further include a flush valve on the proximal hub configured to simultaneously flush the plurality of internal lumens. The distance the exit port is located proximal to the distal-most end of the elongate body can be at least about 10 mm up to about 30 mm. The elongate body can have a length adapted to be introduced into an access site at a left or right common carotid artery, or a left or right subclavian artery such that the distal-most end is positionable within the descending aorta. The length can be between about 10 cm up to about 50 cm. The proximal hub can include one or more proximal orientation markings that are directly visible by a user. The proximal orientation markings can be on a proximal-facing surface of the hub. At least one of the first and second radiopaque marker bands can include cut-outs separated from one another around a circumference of the distal end region so as to correspond circumferentially to the one or more proximal orientation markings. The cut-outs can form gaps in radiopacity in the marker band creating at least two non-radiopaque distal orientation markings. Overlap of the at least two distal orientation markings can indicate circumferential orientation of the distal end region of the elongate body.

In an interrelated aspect, provided is a method of treating a vessel using an arterial access sheath having an elongate body having a plurality of internal lumens and a proximal hub. The method includes inserting the elongate body through a penetration in a wall of a vessel of a patient so that the proximal hub is accessible from outside the patient; inserting a first device into a first proximal opening into a first lumen of the plurality of internal lumens through the proximal hub and out a distal opening from the first lumen; and inserting a second device into a second proximal opening into a second lumen of the plurality of internal lumens through the proximal hub and out an exit port from the second lumen. The first lumen and the second lumen have different internal diameters. The distal opening from the first lumen is located at a distal-most end of the elongate body and the exit port from the second lumen is located through a sidewall of the elongate body a distance proximal to the distal-most end.

The elongate body can include at least a first radiopaque marker embedded within a distal end region of the elongate body near the distal opening and a second radiopaque marker embedded within a region of the elongate body near the exit port. The region of the elongate body having the second radiopaque marker can be located adjacent a proximal side of the exit port, adjacent a distal side of the exit port, or along a circumference incorporating the exit port. The first device can include an endovascular valve delivery system and the second device can include an embolic protection device. The proximal hub can include a plurality of proximal markers providing information about the plurality of internal lumens. The information provided can include at least one of lumen size, distal opening location, and purpose of lumen.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with references to the following drawings. Generally speaking the figures are exemplary and are not to scale in absolute terms or comparatively but are intended to be illustrative. Relative placement of features and elements is modified for the purpose of illustrative clarity.

FIG. 1 shows a side view of an implementation of an access sheath;

FIG. 2A shows a distal end region of the access sheath of FIG. 1;

FIG. 2B shows a proximal end view of the access sheath of FIG. 1;

FIG. 3A shows a proximal end view of an access sheath having a series of proximal orientation markings;

FIGS. 3B-3D show the distal end region of the access sheath of FIG. 3A in different stages of rotation illustrating an arrangement of markings on a radiopaque marker band;

FIGS. 4A-4C show a distal end region of an access sheath in different stages of rotation illustrating another arrangement of markings on a radiopaque marker band;

FIGS. 5A-5C show a distal end region of an access sheath in different stages of rotation illustrating another arrangement of markings on a radiopaque marker band;

FIGS. 6A-6C are cross-sectional view of the elongate body of sheaths having various non-circular shapes;

FIG. 7A shows an implementation of a transcarotid prosthetic aortic valve and delivery system;

FIG. 7B shows another implementation of a transcarotid prosthetic aortic valve and delivery system;

FIG. 7C shows circumferential alignment of an access sheath having distal orientation markings and a valve delivery system having distal orientation markings;

FIGS. 8A-8D show various stages of circumferential alignment of the access sheath of FIG. 5A relative to the 3-cusp line of the aortic valve;

FIG. 9A shows a side view of an access sheath;

FIG. 9B shows a proximal end view of the access sheath of FIG. 9A;

FIG. 9C shows a perspective view of the proximal end region of the access sheath of FIG. 9A;

FIG. 10A shows a perspective view of a distal end region of the access sheath of FIG. 9A;

FIG. 10B shows a distal end view of the access sheath of FIG. 9A;

FIG. 10C shows the access sheath of FIG. 9A inserted via left common carotid artery with a distal end region within the aorta and a side exit oriented towards the right carotid artery.

### DETAILED DESCRIPTION

The systems described herein allow arterial access, such as transcarotid access via the common carotid artery, or subclavian access via the subclavian artery, or transfemoral access via the femoral artery, to the native aortic valve for implantation of devices such as prosthetic aortic valves into the heart or aorta. The systems maximize physician efficiency, control and deployment accuracy of transcatheter aortic valve replacement (TAVR) of a diseased valve, for example, by providing circumferential, axial, and/or radial alignment of the system relative to the anatomy and/or unique lumens for various components to be deployed.

As used herein, “circumferential” alignment or position refers to an alignment or position that is generally around a longitudinal axis of a system. For example, an access sheath 110 may have a longitudinal axis extending through its lumen between a proximal opening into the lumen and a distal opening from the lumen. Circumferential alignment of the distal end region of the access sheath 110 refers to a relative rotation of the distal end region of the access sheath 110 around the longitudinal axis in a circumferential direction of the tube. Circumferential alignment may be used interchangeably with rotational alignment, for example. As used herein, “axial” alignment or position refers to an alignment or position that is generally along the longitudinal axis of the system. Axial alignment of the distal end region of the access sheath 110 refers to a relative extension of the sheath 110 through the vessel or along or in a direction of the longitudinal axis of the system. An access sheath 110 that is adjusted longitudinally is moved in an axial direction along the longitudinal axis, generally, further distally or proximally relative to the anatomy it is inserted through. Axial alignment may be used interchangeably with longitudinal alignment. As used herein, “radial” alignment or position refers to an alignment or position that is at an angle to the longitudinal axis of the system. Radial alignment of the distal end region of the access sheath 110 refers to a relative tilting of the distal end region relative to the longitudinal axis and from a particular fluoroscopic view.

Although certain figures may show the sheath inserted via a particular access site (e.g., the common carotid artery), a similar sheath or sheath/shunt systems may be designed for subclavian access or transfemoral access. Although the sheath 110 is described in the context of delivery of heart valves, the orientation registration between the access sheath 110 and a tool being inserted through the lumen 120 of the sheath 110 can be useful for other sorts of treatments including treatments of the coronary arteries, renal arteries hepatic arteries, thoracic aortic aneurysms, and abdominal aortic aneurysms, and others.

Transfemoral, transcarotid, or subclavian access to the aortic valve can be accomplished via a percutaneous puncture or direct cut-down to the artery. A cut-down may be advantageous due to the difficulty of percutaneous vessel closure of larger arteriotomies, particularly in the common carotid artery. If desired, a pre-stitch may be placed at the arteriotomy site to facilitate closure at the conclusion of the procedure. An access sheath with associated dilator and guidewire is provided that can be sized to fit into the access artery. In the case of carotid access, the access sheath is inserted into the artery inferiorly towards the aortic arch. Either the left or the right common carotid or subclavian artery may be selected as the access site, based on factors including, for example, the disease state of the proximal artery and/or the aorta and the angle of entry of the carotid or innominate artery into the aorta. The carotid artery may then be occluded distal to the access site. If the access is via a direct surgical cut-down and arteriotomy, the occlusion may be accomplished via a vascular clamp, vessel loop, or Rummel tourniquet. Alternately, the access sheath itself may include an occlusion element adapted to occlude the artery, for example an occlusion balloon, to prevent embolic particulates from entering the carotid artery distal to the access site during the procedure.

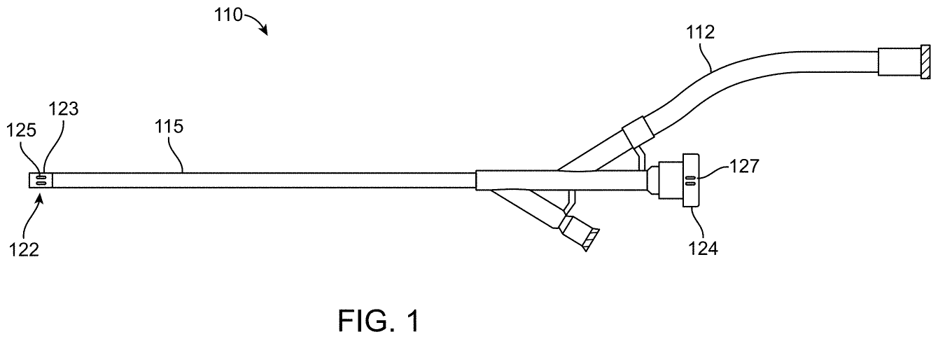

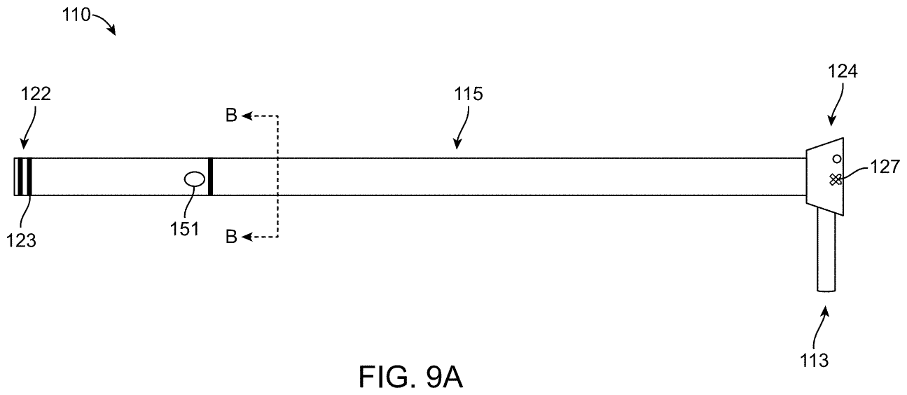

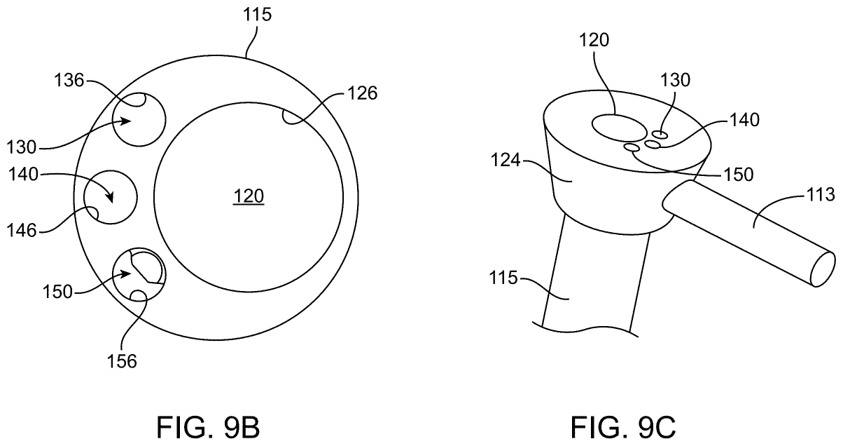

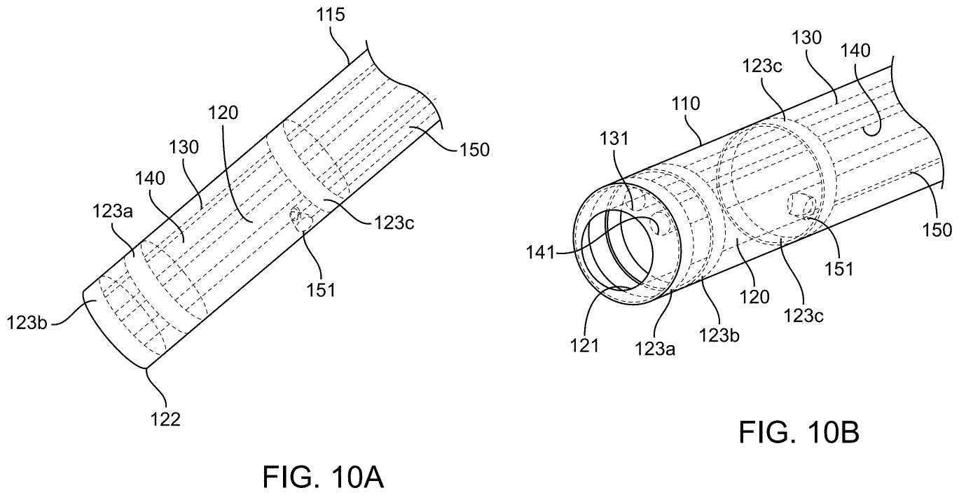

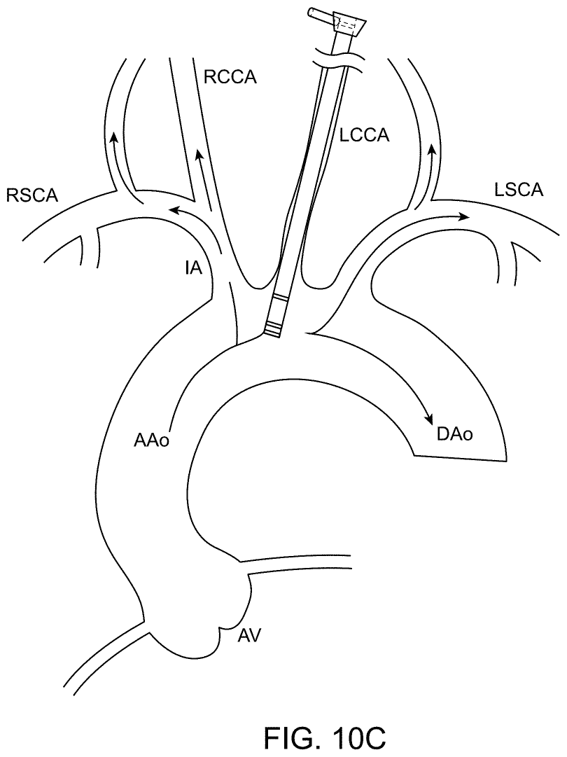

FIG. 1 shows a side view of an implementation of an arterial access sheath 110 formed of an elongate body 115 having an internal lumen 120. Although the sheath 110 is shown having a single internal lumen 120, the sheath 110 can incorporate multiple internal lumens as described below with respect to FIGS. 9A-9C and FIGS. 10A-10C described in detail below. Where the sheath 110 is described as having a single lumen, the same features may be incorporated within a sheath 110 having multiple lumens 120.

The elongate body 115 can be a thin-walled polymer tube having an atraumatic distal tip 122 and a proximal hub 124 to allow for hemostasis and device insertion. The distal end region of the elongate body 115 can incorporate a radiopaque marker band 123 having a plurality of orientation markings 125 to signify circumferential alignment of the distal end region of the sheath 110 under fluoroscopy. A proximal end region of the sheath 110 that is intended to remain outside the patient during use, such as the proximal hub 124, can additionally incorporate one or more orientation markings 127. The proximal orientation markings 127 can coordinate or align circumferentially with the distal orientation markings 125 in a manner that provides detectable guidance to a user with regard to circumferential and longitudinal alignment of the distal end region of the sheath 110. The orientation markings 125, 127 on the sheath 110 can also coordinate or align circumferentially with an interventional tool or delivery system used with the sheath 110, such as an implantable heart valve and delivery system, so that the circumferential position of the tool is more easily and clearly understood during use.

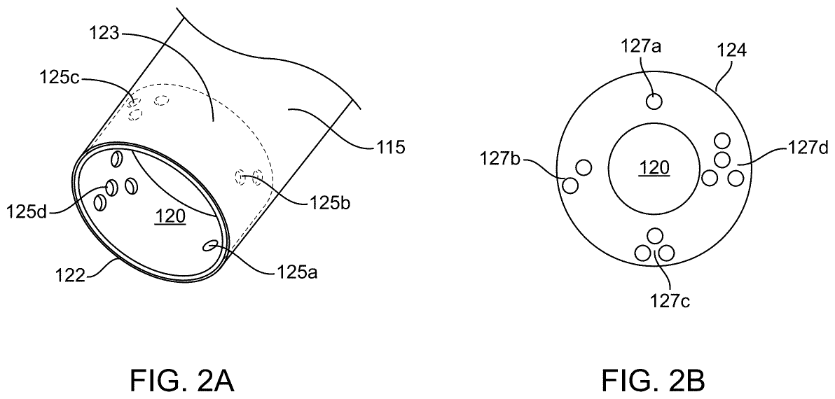

FIG. 2A shows a perspective detail view of the distal end region of the access sheath 110 including the radiopaque marker band 123 having a plurality of orientation markings 125. The markings 125 can be arranged around a circumference of the wall of the elongate body 115 in a plurality of groups that can be distinct from one another so that the markings 125 provide information about the circumferential orientation of the distal end region. FIG. 2B shows a proximal end view of the proximal end hub 124 of the access sheath 110 and the plurality of orientation markings 127. As with the distal markings 125, the proximal markings 127 can be arranged around a circumference of the tool, for example on a proximal-facing surface of the hub 124. The proximal markings 127 can be arranged in a plurality of groups that coordinate with the distal markings 125 at the distal end region of the sheath 110 to provide information about the circumferential orientation of the distal end region of the sheath 110. Thus, if the distal end region includes a first group of markings 125 including a single discernable shape such as a single circle (or square, rectangle, triangle, diamond, arrow, hemisphere, or other shape), so too does the first group of markings 127 on the proximal hub 124. Whatever arrangement of marking is provided at the distal end region of the sheath, the markings 127 at the proximal hub 124 can too. As an example, FIGS. 2A-2B show a first group of distal markings 125a and a first group of proximal markings 127a that can include a single discernable shape or number of markings (e.g., a single circle, square, rectangle, triangle, diamond, arrow, hemisphere, or other shape) and can be located at a distinct location around a circumference of the elongate body wall. A second group of distal markings 125b and a second group of proximal markings 127b can include two discernable shapes or markings (e.g., two circles, squares, rectangles, triangles, diamonds, arrows, hemispheres, or other shapes) located a distance around the circumference. A third group of markings 125c and a third group of proximal markings 127c can include three discernable shapes or markings (e.g., 3 circles) and be located a further distance around the circumference. A fourth group of markings 125d and a fourth group of proximal markings 127d can include another number of discernable shapes or markings (e.g., 4 circles) and be located a further distance around the circumference.

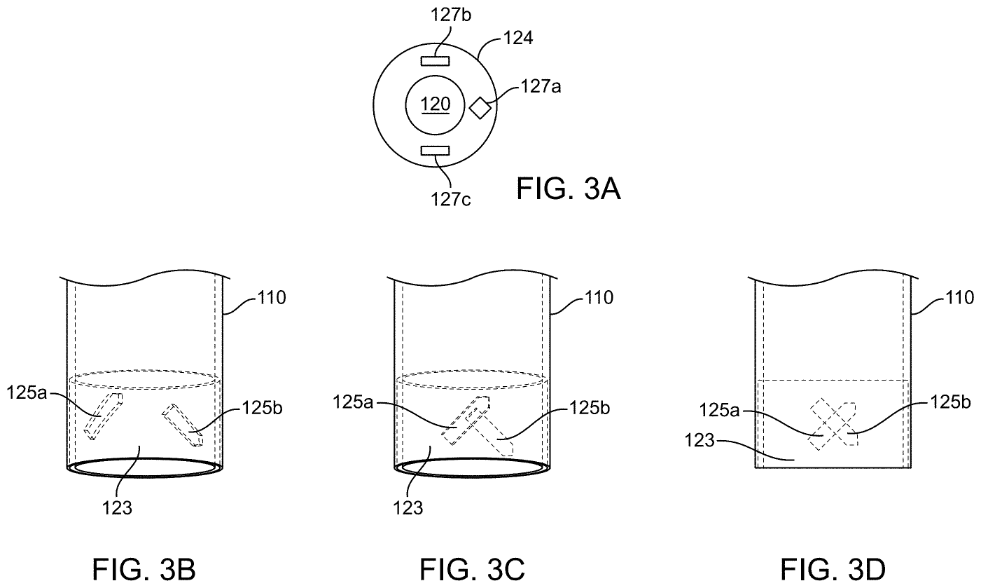

Any of a variety of markings or groups of markings 125, 127 around the circumference is considered so long as the markings or groups of markings 125, 127 are distinguishable from one another. Similarly, any of a variety of shapes or number of shapes within a particular group of markings 125 is considered including circle, square, rectangle, triangle, diamond, arrow, hemisphere, or other shape. For example, FIG. 3A illustrates another series of distinct markings 127 on the proximal hub 124 where each “group” is a single marker having a particular distinguishable shape. A “front” of the sheath 110 can be identified by a first shaped marker 127a (e.g., a diamond) and the “sides” of the sheath 110 relative to that first marker 127a can be identified by a second shaped marker 127b, 127c. The distal markings 125 need not match exactly the shape, size, count of the proximal markings 127. For example, FIGS. 3B-3D show a distal end region of the sheath of FIG. 3A in different stages of rotation. Despite having three proximal markings 127, there are shown just two markings 125a, 125b separated from one another around a circumference of the radiopaque marker band 123. The first marking 125a can be an arrow or other sort of non-symmetrical shape that is distinct from the shape of the second marking 125b. FIG. 3B shows the first and second markings 125a, 125b out of overlapping alignment with one another and FIG. 3C shows them in partial alignment and overlapping in part. FIG. 3D shows the first and second markings 125a, 125b in full alignment with one another so that the rectangular arrow shapes are fully overlapped and creating a symmetrical x-shape. The arrangement of the asymmetrical markings 125a, 125b when fully overlapping and aligned with one another provides a further message to a user due to their alignment, which is where the “front” of the catheter is in space around the longitudinal axis. One of the markings 127a of the proximal hub 124 can also indicate this desired side. Knowing where a particular side or “front” of a catheter is becomes important to a user when deploying an asymmetrical tool or device through the catheter as will be described in more detail below.

As used herein, “fully overlapping” refers to the circumferential rotation of the distal end region of the access sheath 110 around the longitudinal axis of the catheter and the radial angle of the catheter relative to the longitudinal axis that results in a pair of markings 125 being substantially aligned with one another relative to a view of choice on fluoroscopy. For example, if a pair of markings 125a, 125b are formed as rectangular openings or cut-outs in the radiopaque band 123, when the markings 125a, 125b are fully overlapped, all four sides of the rectangle of one of the markings 125a are substantially aligned with all four sides of the rectangle of the other of the markings 125b so that only a single rectangle shape is visible under fluoroscopy (e.g., as an absence of or gap in radiopacity in an otherwise radiopaque distal end region) when viewed from a traditional three-cusp coplanar view or right/left cusp-overlap view. The angiographic projection is often perpendicular to the virtual plane of the aortic annulus. In traditional three-cusp view all three cusps are visible, generally corresponding to a left anterior oblique cranial (LAO-CRA) view. In the right/left cusp-overlap view, the right coronary cusp (RCC) and the left coronary cusp (LCC) are superimposed on top of each other either using multislice CT (MSCT) or angiography, generally corresponding to a right anterior oblique caudal (RAO-CAU) view, leaving the non-coronary cusp (NCC) isolated. This creates a two-cusp view or cusp overlap view. The cusp overlap view provides advantages in terms of deployment depth at the level of the NCC, the position and tension of the wire can be assessed. Additionally, the right/left cusp-overlap view allows the operator to definitively predict the spatial orientation of the native valve commissures as one of these three commissures is located between the right coronary cusp and the left coronary cusp. Overlapping of the non-radiopaque marking allows for accurate alignment of the sheath to a native commissure. The access sheath 110 and delivery systems appear substantially vertical in this view.

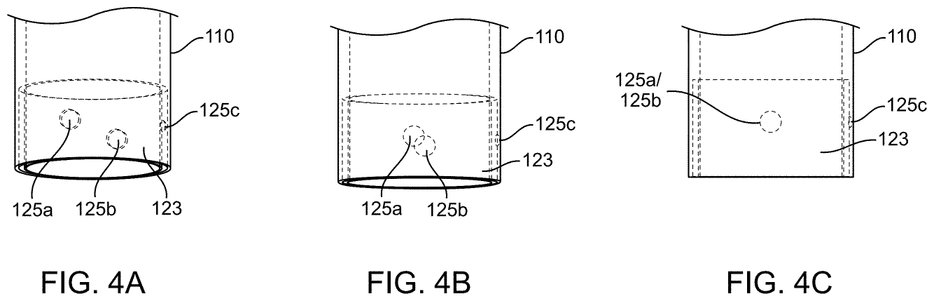

FIGS. 4A-4C show another arrangement of markings 125 on the radiopaque marker band 123 that depending on their overlapping arrangement provides information to the user about circumferential positioning. The marker band 123 can include a pair of markings 125a, 125b separated from one another around a circumference of the band 123. The marker band 123 can additionally incorporate a marking 125c that is unique from the other two markings to identify the “front” of the access sheath 110. The third marking 125c can be a small projection or button of radiopaque material projecting away from the side wall of the sheath that is visible on fluoroscopy. FIG. 4A shows the first and second markings 125a, 125b out of alignment with one another and FIG. 4B shows them in partial alignment and overlapping in part. FIG. 4C shows the first and second markings 125a, 125b in full alignment with one another so that they no longer appear as two markings, but a single marking indicating full circumferential alignment. The third marking 124c is then positioned and visible at the “front” of the catheter.

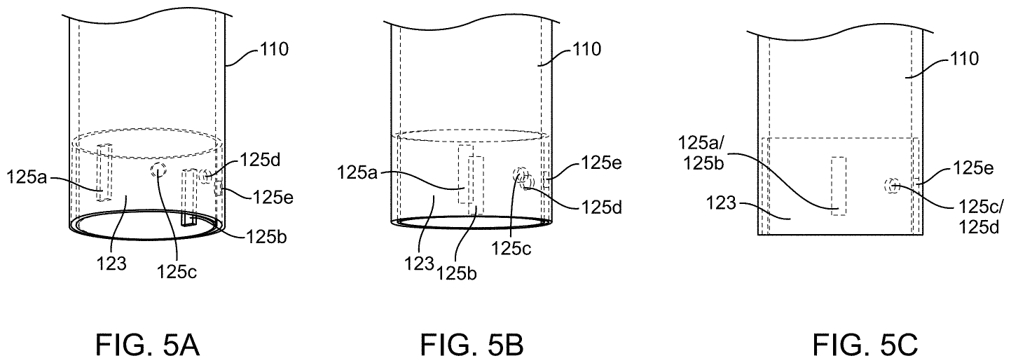

FIGS. 5A-5C show another arrangement of markings 125 on the radiopaque marker band 123 that depending on their overlapping arrangement provides information to the user about circumferential positioning. The marker band 123 can include a first pair of markings 125a, 125b that are similar to one another in shape and size yet separated around a circumference of the catheter. The marker band 123 can include a second pair of markings 125c, 125d that are also similar to one another in shape and/or size, separated around a circumference of the catheter. The second pair of markings 125c, 125d, however, can be unique from the first pair of markings 125a, 125b in shape and/or size so that upon alignment of the pairs the “front” of the catheter is identified. FIG. 5A shows all the markings out of alignment with one another. FIG. 5B shows them in partial alignment and overlapping in part. FIG. 5C shows the first pair of markings 125a, 125b aligned with one another and the second pair of markings 125c, 125d aligned with one another. A fifth marking 125e can be unique from the other two pairs of markings to identify the “front” of the access sheath as described above.

Regardless the arrangement or configuration, the distal orientation markings 125 and the proximal orientation markings 127 coordinate in a way obvious to the user to provide information regarding the circumferential alignment of the distal end region of the access sheath 110 under fluoroscopy and guided by the proximal orientation markings 127 visible at or near the hub of the sheath 110. This coordination is useful for delivering an interventional device through the sheath where the device has “handedness” or some sort of asymmetry that involves implanting and/or delivery according to a particular orientation.

The sheath 110 can be used to deploy any of a variety of devices that require precise positioning within the vasculature according to an appropriate circumferential orientation including prosthetic valves, differentially porous stents having asymmetrical braiding or coils that create areas of lesser or greater blood flow, fenestrated or branched devices, flow diverters configured to divert blood flow away from an aneurysm, fistula, or ruptured vessel may have a region that allows for flow to healthy tissue, vascular occluding devices that incorporate asymmetrical braids or differential lattice densities, and others.

The radiopaque marker band 123 can be created from polymer of the catheter that is loaded with one or more radiopaque materials including tungsten, tantalum or barium sulfate, platinum, stainless steel, or gold. The distal orientation markings 125 can be created as a cut-out forming an absence of these radiopaque materials in the radiopaque marker band 123 that is detectable as having a unique contrast under fluoroscopy relative to the band 123. There are two significant advantages of the markings 125 being created as an absence of a radiopaque material. One is that the relatively large radiopaque marker band 123 is easily visible under angiography (location and distal edge), which is important for safe tracking of the device through the vessel. Another is that laser cutting or machining of the cut-outs in radiopaque material allows for accurate spatial orientation between the markings during manufacture of the device (as opposed to adhering multiple orientation markers/components to a polymeric shaft in a controlled pattern). The markings 125 can be formed into the band 123 as cut-outs forming one or more gaps in the radiopacity of the band 123 that appears as an elongate slot, cut-out, hatch mark, hole, channel, or other region in the polymer material of the sheath 110 that lacks radiopaque material. Thus, the markings 125 are fully polymeric without any radiopaque material so that, unlike the remainder of the radiopaque band 123, appear on fluoroscopy as distinct regions of light within an otherwise dark band. In some implementations, the marking or pair of markings 125 is formed as a non-radiopaque slot within the radiopaque band 123 that is in a shape of an elongate rectangle. In other implementations, the marking or pair of markings 125 is formed as a non-radiopaque aperture within the radiopaque band 123 that is circular in shape. In still other implementations, the marking or pair of markings 125 is formed as a non-radiopaque cut-out formed within the band 123 having shapes that are unique from one another (upward and downward arrows) that, upon overlapping one another, appear to create a new marking with a different singular shape (X-shape). The distinct region formed by the marking 125 can have any of a variety of shapes including geometric and free-form shapes. Alternatively or additionally, the distal orientation markings 125 can be a different material from the material of the marker band 123 so that they appear as a unique mark having a unique contrast under fluoroscopy relative to the band 123. The different material can also be radiopaque, but can be thinner than a remainder of the band 123 so that the markings do not appear to be as dark as the remainder of the band 123. Thus, the markings 125 can have a different radiopacity than a radiopacity of the band 123 the difference being discernible under fluoroscopy.

The proximal markings 127 need not be radiopaque as they are arranged on a region of the sheath 110 that is external to the patient so they are visible to the naked eye during a procedure. The proximal markings 127 can be applied according to any of a variety of known methods including sticking as if by an adhesive sticker marking, painted marking, molded marking, etched marking, embossed marking, printed marking, or otherwise applied to a region of the sheath 110 external to the patient, such as the proximal hub 124, so that they are readily visible by a user during a procedure.

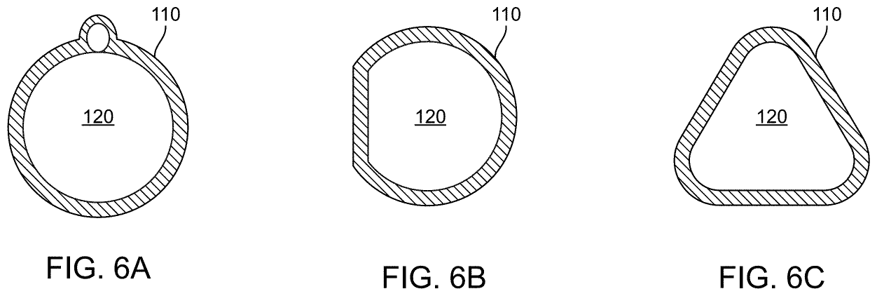

The elongate body 115 of the sheath 110 can be circular in cross-section or can be non-circular in cross-section. A non-circular cross-section of the elongate body 115 of the sheath 110 allows for the sheath to be keyed to another component. The keyed mating can coordinate with the circumferential alignment of the distal and proximal markers 125, 127. FIGS. 6A-6C show various non-circular cross-sections of the elongate body 115 of the sheath 110. In some implementations, the inner and outer dimensions of the sheath 110 are non-circular whereas in others only the inner dimension or outer dimension of the sheath 110 is non-circular. In other words, the outer diameter of the sheath 110 can be circular whereas the inner diameter of the sheath 110 can be non-circular or vice versa. The cross-section, whether the entire cross-sectional shape of the sheath 110 or just the cross-sectional shape of the lumen 120, may include flat polygonal shapes or free-form shapes with only a particular orientation. The cross-sectional shape can be oval, D-shaped, triangular, rectangular, or other non-circular shape. The sheath 110 can additionally or alternatively incorporate a rail feature (see FIG. 6A). The elongate body 115 including an asymmetry around its circumference can incorporate proximal orientation markings that correspond to the asymmetry.

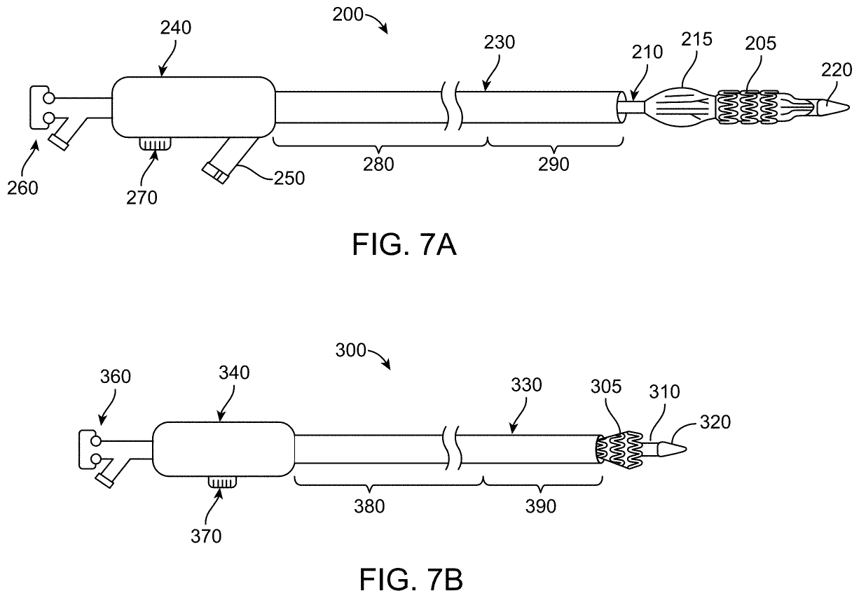

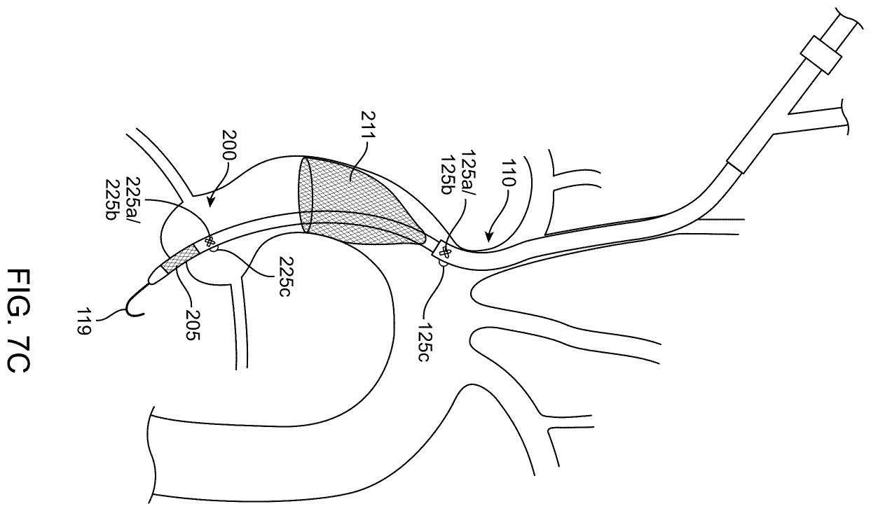

The keyed cross-section of the access sheath can be designed to receive a similarly keyed cross-section of an endovascular delivery system so that the cross-section is circumferentially aligned to the implantable device to be deployed. For example, a TAVR delivery system deployed through the lumen 120 can have an outer dimension correspondingly shaped or keyed to the cross-sectional shape of the lumen 120 to provide circumferential alignment to the implantable heart valve (see FIG. 7A-7B). The delivery system can be inserted so that the valve is aligned relative to the proximal markings 127 on the access sheath 110 and so that the corresponding shapes of the inner diameter of the sheath 110 and the outer diameter of the delivery system match to achieve the desired orientation at the distal end region of the sheath 110.

The markers 125, 127 and the non-circular cross-section of the sheath body 115 can provide a reproducible positioning that provides accurate and appropriate orientation of the valve being delivered.

In some implementations, the hub 124 incorporates an overall shape such as non-circular or polygonal shape or incorporate a registration feature such as a bump-out or rail along a particular orientation that can visually and/or physically align to the distal orientation markings 125 on the distal end region of the access sheath 110. Thus, the hub 124 need not incorporate proximal orientation markings 127 per se, but rather can take on a shape that provides the same sort of information the proximal orientation markings 127 provide. The shaped hub 124 can also provide keyed alignment with one or more devices being inserted through the hub 124 so that circumferential orientation of the device is known and predictable.

The hub 124 can incorporate a silicone slit valve or luer attachment to connect Touhy-Borst style. The hub 124 of the arterial access sheath 110 may include a Y-arm for delivery of contrast or saline flush, for aspiration, and/or may be fluidly connected to a shunt 112, wherein the shunt provides a shunt lumen or pathway for blood to flow from the arterial access sheath 110 to a return site such as a venous return site or a collection reservoir. In this regard, a retrograde or reverse blood flow state may be established in at least a portion of the artery. The Y-arm for inflation of an occlusion balloon on a distal end region of the sheath, if present, via an inflation lumen, and a hemostasis valve for introduction of an endovascular valve delivery system into the sheath.

The sheath 110 can have a working length (i.e., the portion of the sheath that is insertable into the artery during use) that varies to accommodate a wide range of devices having various sizes. The working length can be about 10 cm-50 cm. In implementations, where the sheath 110 incorporates a single internal lumen 120, the lumen of the sheath can have an inner diameter large enough to accommodate insertion of an endovascular valve delivery system, such as an 18 French to 22 French (0.236″ to 0.288″) system. In other implementations, wherein the sheath 110 incorporates a plurality of internal lumens, the lumen size can vary as discussed in more detail below. The sheath size can be as small as about 4 French up to about 24 French. In an implementation, the delivery system has an inner diameter as small as about 0.182″.

In an implementation, the access system may be equipped with one or more embolic protection elements to provide embolic protection for one or both carotid arteries. For example, a filter may be included in the access system to provide embolic protection for one or both carotid arteries. In a variation of this implementation, the filter is deployed via the contralateral carotid, brachial or subclavian artery, and positioned in the aortic arch across the ostium. If the sheath access site is the left common carotid artery, the filter may be positioned across the ostium of the innominate (also known as brachiocephalic) artery. If the sheath access site is the right common carotid artery, the filter may be positioned across the ostium of the left common carotid artery. In a variation of this implementation, the filter is deployed across both the innominate and left common carotid artery, or across all three head and neck vessels (innominate artery, left common carotid artery, and left subclavian artery). The filter element may be built-in to the access sheath 110. Or, the filter element may be a separate element which is compatible with the access sheath 110. For example, the filter element may be a coaxial element that is slideably connected to the access sheath or an element which is placed side-by-side with the access sheath. The filter element may comprise an expandable frame, so that it may be inserted into the artery in a collapsed state, but then expanded at the target site to position the filter element across the opening of the artery or arteries. The embolic protection provided by the sheath can include any of the systems described in U.S. Pat. Nos. 8,545,552 and 10,925,709; or PCT Publication No. WO 2021/087480, which are each incorporated by reference herein.

An endovascular valve delivery system including a prosthetic valve and a delivery catheter may be advanced through the access sheath 110. The delivery system can be capable of torsional control and incorporating distal and proximal markings similar to the access sheath and as described above. The various configurations of distal and proximal orientation markings described above with respect to the access sheath 110 are considered herein with respect to the endovascular valve delivery system. The coordinated distal and proximal markings of the delivery system provide the user with information regarding the circumferential orientation of the implantable device in reference to the delivery system and also in reference to the access sheath through which the device is delivered. This allows for the user to circumferentially position the sheath to a desired alignment within the patient using fluoroscopy guidance and insert the endovascular valve delivery system along the guided pathway to deliver the implantable device with a known circumferential position.

An exemplary valve and delivery system configured to be delivered through an access sheath 110 described herein is shown in FIG. 7A. FIG. 7A shows an implementation of a balloon expandable prosthetic aortic valve 205 mounted on the distal end of an endovascular valve delivery system 200. The delivery system can have a distal tapered tip 220 and an expandable balloon 215 on the distal end region of an inner shaft 210. In an implementation, the system also has an outer sleeve, such as for example a pusher sleeve 230, that is slidable along the long axis of the device and that maintains the valve in position on the balloon during delivery. A proximal control assembly contains a mechanism for retracting the pusher sleeve 230, such as a sliding button 270 on a proximal handle 240. In FIG. 7A, the pusher sleeve 230 is shown retracted from the valve 205 and proximal balloon 215 so that the valve 205 can be expanded without interference from the pusher sleeve 230. A connector 250 allows connection of an inflation device to the balloon inflation lumen of the balloon 215. A proximal rotating hemostasis valve 260 allows system flushing as well as sealing around a guidewire (not shown) as the valve delivery system 200 is being advanced over the guidewire and into position.

The delivery catheter can have a working length of 30, 40, 60, 70, or 80 cm up to about 110. In an implementation, the delivery catheter has a working length designed for transcarotid access and has a working length of between about 30 cm and about 50 cm. In an implementation, the delivery catheter has a working length designed for transfemoral access and is about 110 cm. The route from the transcarotid access site is fairly short and straight, as compared to the transfemoral or subclavian approach. As a result, the delivery system for deployment through an access sheath positioned in the carotid artery can be shorter than one for deployment through a transfemoral access sheath and the proximal section can be quite rigid, both of which will allow greater push and torque control resulting in increased accuracy in positioning and deploying the prosthetic valve. The distal section has increased flexibility to allow accurate tracking around the ascending aorta and into position at the aortic annulus. Materials for the delivery system may include reinforced, higher durometer, and/or thicker walled materials as compared to current delivery systems to provide this increased rigidity.

In an implementation, the working length of the valve delivery system 200 is configured to allow delivery of the valve to the aortic annulus from a transcarotid access site. Specifically, the working length of the valve delivery system 200 is between 45 and 60 cm. The delivery system shaft can also be configured for delivery from a right or left carotid access site. Specifically, the shaft has a proximal stiff section 280 and a more flexible distal section 290. In an implementation, the distal section 290 is 2 to 4 times more flexible than the proximal stiff section 280. In an implementation, the distal flexible section 290 is between one quarter to one third the total working length of the valve delivery system 200. Specifically, the distal flexible section 290 can be in the range 10 cm to 20 cm. In an alternate implementation, the valve delivery system 200 has a transition section of one or more flexible lengths which fall between the flexibility of the distal flexible section 290 and the proximal stiff section 280.

Another implementation of a valve and delivery system 300 configured for transcarotid delivery is shown in FIG. 7B. The self-expanding prosthetic aortic valve 305 is mounted on the distal end of an endovascular valve delivery system 300. The delivery system has a distal tapered tip 320 on the distal end of an inner shaft 310. The valve 305 is positioned on the inner shaft 310 and contained in a retractable sleeve 330 that can slide along the longitudinal axis of the device. A proximal control assembly contains a mechanism for retracting the retractable sleeve 330, such as a sliding button 370. In an implementation, the design of the valve 305 and sleeve 330 are such that the sleeve 330 can be re-advanced in a distal direction to abut and to collapse the valve 305 so that the valve 305 can be re-positioned if the first position was inaccurate. A proximal rotating hemostasis valve 360 allows system flushing as well as sealing around a guidewire (not shown) as the valve delivery system is being advanced over the guidewire and into position.

As with the previous embodiment, the working length of the valve delivery system 300 is configured to allow delivery of the valve 305 to the aortic annulus from a transcarotid access site. Specifically, the working length of the valve delivery system 300 is between 45 and 60 cm. The delivery system shaft is also configured for delivery from a right or left carotid access site. Specifically, the shaft has a proximal stiff section 380 and a more flexible distal section 390. In an embodiment, the distal section is 2 to 4 times more flexible than the proximal stiff section. In an embodiment, the distal flexible section is between one quarter to one third the total working length of the valve delivery system. Specifically, the distal flexible section is in the range 10 cm to 20 cm. In an alternate embodiment, the valve delivery system has a transition section of one or more flexible lengths which fall between the flexibility of the distal flexible section and the proximal flexible section.

TAVR requires accurate placement of the valve either into the diseased native valve, or into a previously implanted valve. Longitudinal and circumferential alignment of the new valve is critical to achieve procedural success. Alignment is critical to correct placement of apposition and not interfering with blood flow to the coronary arteries. Clinically, the circumferential alignment of the valve posts and leaflets relative to the native or implanted aortic valve commissures is also necessary for a successful procedure. The orientation markings 125, 127 and/or keyed arrangement of the proximal hub, and/or sheath registration features provide guidance regarding proper alignment of the sheath to the anatomy. The delivery system 200 (or 300) can incorporate one or more distal orientation markings 225 as well as one or more corresponding proximal orientation markings that provide visual guidance as to the orientation of the valve 205 (or valve 305) positioned relative to the delivery system 200. The distal and proximal orientation markings can be of the same character and function as the orientation markings 125, 127 described above with respect to the access sheath 110. FIG. 7C illustrates an implementation of an access sheath 110 having an embolic protection filter 211 on a distal end region and a valve delivery system 200 extending through it. The distal end region of the access sheath 110 is shown with distal markings 125a/125b aligned with one another forming an X-shape and a third distal marking 125c projecting from a side of the distal end region of the sheath 110. The delivery system 200 is shown having a valve 205 mounted on a distal end region and advanced over a guide wire 119. The distal end region of the delivery system 200 can include corresponding distal orientation markings 225a/225b forming an X-shape and a third distal marking 225c projecting from the same side of the distal end region of the delivery system 200 as the third distal marking 125c projects from the sheath 110 indicating proper alignment around the longitudinal axis for deployment within the target anatomy.

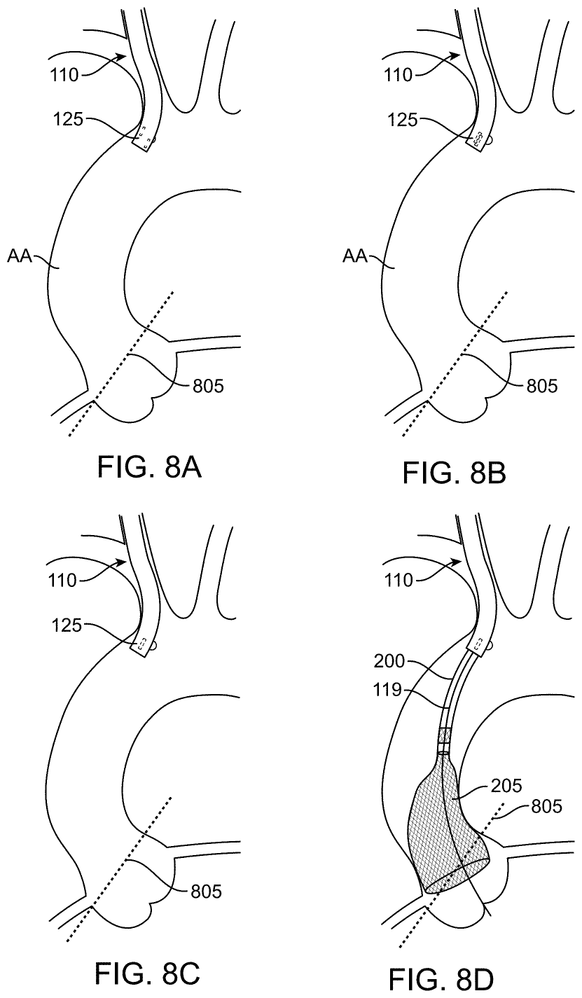

FIGS. 8A-8D illustrate various stages of circumferential alignment of an access sheath 110 relative to the three-cusp line 805 of the aortic valve including the non-coronary cusp (NCC), right coronary cusp (RC), and left coronary cusp (LC) when viewed under fluoroscopy, representative of the right/left cusp-overlap view. FIG. 8A shows the distal end of the sheath 110 positioned within the ascending aorta AA. The distal orientation markings 125 of the radiopaque band 123 are misaligned. The configuration of the distal orientation markings 125 are similar to those shown in FIGS. 5A-5C having two alignment slits and two front circles as well as a fifth marking indicating a “front”. FIG. 8B shows that the access sheath 110 has rotated around the longitudinal axis of the elongate body so that the distal orientation markings 125 partially align. FIG. 8C shows that the access sheath 110 has rotated even further around the longitudinal axis of the elongate body so that the distal orientation markings 125 are fully aligned with one another. FIG. 8D shows valve delivery through the aligned access sheath 110. The distal orientation markings 125 of the access sheath can indicate a particular direction of the patient. Two markings 125 can overlap creating a single mark on the distal end of the sheath 110 that result in another marking 125 directing toward a particular part of the patient (e.g., facing anterior of patient) so that upon insertion of the implantable device, the device can be oriented properly relative to the target site, in the case of FIG. 8D is the aortic valve. The implantable device can be inserted through the sheath so that commissures of the implant are aligned with the proximal orientation markings 127 that coordinate with the distal orientation markings 125 identified under fluoro. The distal orientation markings 125 can be properly oriented to the anatomy under fluoroscopy and the proximal orientation markings 127, which are visible outside the patient and coordinate directly to the distal orientation markings 125, can be used to guide proper insertion of the implant delivery system into the hub 124 of the sheath 110. The delivery system for the implant can additionally incorporate one or more features to ensure the proper alignment during insertion of the system is achieved.

The access sheath and delivery system features described herein are useful for TAVR as well as a variety of other interventional methodologies where accuracy of implantation and orientation of device deployment is desirable, for example, in the treatment of coronary arteries, renal arteries, hepatic arteries, thoracic aortic aneurysms, and abdominal aortic aneurysms. Although the methods described herein are described in the context of TAVR, it should be appreciated that the sheaths described herein can be used for any of a variety of methods.

In an implementation, the access sheath 110 is first inserted into the vasculature such as via either a percutaneous puncture or direct surgical cut-down and puncture of the carotid artery. The access sheath 110 can be introduced through the penetration with the tip directly inferiorly towards the ostium of the artery. A transcarotid approach to the aortic valve may be achieved via the LCCA or via the RCCA.

The distal end region of the access sheath 110 can be aligned to the right/left cusp-overlap view (see FIG. 8A). The sheath 110 can be rotated around the longitudinal axis until the distal orientation markings 125 indicate the proper orientation relative to the anatomy while viewed under fluoroscopy. For example, at least some of the distal orientation markings 125 can overlap and another distal orientation marking 125 can indicate a particular direction or anatomy of the patient (see FIGS. 8B-8C).

Once the access sheath is positioned and optionally the embolic protection means are deployed via occlusion, aspiration, and/or filter elements, access to the aortic valve is obtained via a guidewire 119 (such as a 0.035″ or 0.038″ guidewire) inserted into the sheath 110 and directed inferiorly into the ascending aorta and across the native aortic valve. Pre-dilation of the native aortic valve can be performed with an appropriately sized dilation balloon, for example a valvuloplasty balloon, before valve implantation. The guidewire 119 is used to position a balloon across the valve and the balloon is inflated, deflated, and then removed while the guidewire remains in place.

An endovascular prosthetic valve 205 and delivery system 200 is then inserted through the access sheath 110 over the guidewire 119. The valve 205 and delivery system 200 can be introduced so that the commissure is oriented to the proximal orientation markings 127 on the sheath 110, which in turn, correspond directly to the distal orientation markings 125 of the sheath 110. The valve 205 positioned on the delivery system 200 can be tracked through the access sheath 110 to the site of the native aortic valve. The prosthetic valve 205 can be adjusted if needed longitudinally along the long axis and/or circumferentially around the long axis until the desired alignment is achieved. The prosthetic valve 205 is then deployed at or near the position of the native aortic valve (see FIG. 8D). The approach from the carotid artery can increase the circumferential control of the sheath 110 and the prosthetic to improve accuracy of alignment.

At the conclusion of the implantation step, the implanted prosthetic valve 205 function can be assessed via ultrasound, contrast injection under fluoroscopy, or other imaging means. Depending on the design of the delivery system 200, the prosthetic valve 205 may be adjusted as needed to achieve optimal valve function and position before final deployment. The delivery system 200 and guidewire 119 are then removed from the access sheath 110. After removal of the delivery system 200 and guidewire 119, any embolic protection elements are removed. Aspiration may continue during this time to capture any embolic debris caught in the sheath tip, occlusion element and/or filter elements. Various forms of embolic protection can be deployed including occlusion elements, filters, occlusion balloons, aspiration, and the like.

The access sheath 110 is then removed and the access site is closed. If the access was a surgical cutdown direct puncture, the vessel is closed either via tying off the pre-placed stitch or with manual suturing or with a surgical vascular closure device, as described in more detail below. If the access was percutaneous, percutaneous closure methods and devices may be employed to achieve hemostasis at the access site. In an implementation, the closure device is applied at the site of the penetration before introducing the arterial access sheath through the penetration. The type of closure device can vary.

The access site described above is either the left or right common carotid artery. Other access sites are also possible, for example the left or right subclavian artery or left or right brachial artery. These arteries may require longer and/or more tortuous pathways to the aortic valve but may offer other advantages over a carotid artery access, for example the ability to work away from the patient's head, the ability to avoid hostile neck anatomy such as previous carotid endarterectomy or other cervical surgery or radiation, or less risky in case of access site complication. In addition, carotid artery disease, or small carotid arteries may preclude common carotid artery access. In the case of any of these access sites, occlusion, aspiration, and/or filtering the head and neck vessels during TAVI may increase the speed and accuracy of the procedure, and decrease the rate of embolic complications.

The access sheath 110 can be first inserted into the vasculature such as via either a percutaneous puncture or direct surgical cut-down and puncture of the femoral artery and tracked into the descending aorta, across the arch, and into the ascending aorta. The access sheath 110 can be aligned under fluoroscopy as described above using the cusp-overlap view of choice (e.g., 3-cusp, R/L). The TAVR device can be inserted into the sheath 110 with the flush port on the handle oriented according to practice, such as at 3 o′clock or 12 o′clock. The TAVR device can be tracked through the sheath 110 to the aortic valve. The TAVR delivery device handle can be torqued until proper circumferential alignment is achieved, which may involve retrieval of the TAVR device back into the descending aorta, prior to deploying the valve.

FIG. 9A shows a side view of another implementation of the arterial access sheath 110 having multiple internal lumens. The features of the access sheath 110 described above with respect to FIGS. 1-8D apply to the access sheath 110 shown in FIGS. 9A-9C to 10A-10C. For example, the access sheath 110 of FIGS. 9A-9C to 10A-10C may incorporate the plurality of orientation markings described above that are useful for identifying proper orientation of the sheath 110 to the anatomy under fluoroscopy as described in detail above although may or may not be repeated below with respect to FIGS. 9A-9C and 10A-10C.

The elongate body 115 of the sheath of FIGS. 9A-9C can be a thin-walled polymer tube having an atraumatic distal tip 122 and a proximal hub 124 to allow for hemostasis and device insertion through each of the multiple internal lumens. In an implementation, the elongate body 115 can include at least two internal lumens 120 and 130. In another implementation, the elongate body 115 can include at least three lumens 120, 130, and 140. In a still further implementation, the elongate body 115 can include four lumens 120, 130, 140, and 150. The proximal hub 124 can incorporate a hemostasis device such as a silicone slit valve or other sort of feature for each of the lumens.

The first lumen 120 can have an inner diameter sufficient to receive an interventional device such as a TAVR device, for example. The first lumen 120 can be the largest inner lumen and can be between about 12 Fr (4 mm) up to about 24 Fr (8 mm), or approximately 18 Fr (6 mm). The second and third lumens 130, 140 can be smaller compared to the first lumen 120, for example, between about 3 Fr (1 mm) up to about 9 Fr (3 mm). The smaller second and third lumens 130, 140 can be used for delivery of a diagnostic or pig-tail catheter or other device, contrast agent, therapeutic agents, aspiration, and/or pressure measurement.

In some implementations, the sheath 110 can additionally include a further lumen 150 that is designed to exit through a sidewall of the elongate body 115 rather than the distal-most end of the sheath 110. The lumen 150 can be substantially straight along at least a portion of its length and extend from a proximal opening 156 into the lumen 150 towards the distal end region of the sheath 110 along a longitudinal axis and parallel to lumen 120. The lumen 150 can turn near a distal end region to exit out the sidewall at exit port 151. The exit port 151 of the lumen 150 can be located at least about 10 mm up to about 30 mm away from the distal-most end 122 of the sheath 110 to allow for delivery into the aortic arch to track to target vessels for protection (e.g. contralateral carotid artery). The inner diameter of the lumen 150 can be sized smaller than the first lumen 120, for example, about 5 Fr. The exit port 151 from the lumen 150 can be used to facilitate the delivery of an embolic protection device through the lumen 150.

Embolic protection devices used in combination with TAVR are typically advanced to the contralateral carotid artery, brachial or subclavian artery, and/or positioned in the aortic arch across the ostium. For example, if the sheath access site is the left common carotid artery, the filter may be advanced through the lumen 150, out the exit port 151 and positioned across the ostium of the innominate (also known as brachiocephalic) artery. If the sheath access site is the right common carotid artery, the filter may be positioned across the ostium of the left common carotid artery. In a variation of this implementation, the filter is deployed across both the innominate and left common carotid artery, or across all three head and neck vessels (innominate artery, left common carotid artery, and left subclavian artery). The filter element may include an expandable frame, so that it may be inserted into the lumen 150 in a collapsed state, but then expanded at the target site to position the filter element across the opening of the artery or arteries.

The length and inner diameter of the lumens can be modified for appropriate device compatibility. The inner diameter of the first lumen 120 can be large enough to accommodate insertion of endovascular valve delivery systems or other interventional systems. As discussed above, the sheath 110 can have a working length (i.e., the portion of the sheath that is insertable into the artery during use) that varies to accommodate a wide range of devices having various sizes. The working length can be about 10 cm-50 cm. The working length of the sheath 110 can vary depending on the access site. A sheath adapted to be inserted into the common carotid artery for the purpose of access to the descending aorta, the length of the elongated sheath body 115 can be in the range from 10 cm to about 50 cm, usually being about 20 cm. For a sheath adapted to be inserted via the femoral artery to the descending aorta, the length of the elongated sheath body 115 can be in the range from 30 to 100 cm, usually being from 80 cm. The sheath outer diameter, which can be a combination of the various lumens plus the wall thickness, can be approximately 24 Fr for a TAVR lumen of 18 Fr and ancillary lumens of 3 Fr to 6 Fr.

The distal end region of the elongate body 115 can incorporate one or more radiopaque marker bands 123 to signify sheath position under fluoroscopy. As discussed above, the radiopaque markers 123 can be created from polymer of the catheter that is loaded with one or more radiopaque materials including tungsten, tantalum or barium sulfate, platinum, stainless steel, or gold. In an implementation, the distal-most tip of the elongate body 115 can include a first marker 123 and another region of the elongate body 115 can include a second marker 123 that is unique in size, shape, and/or color from the first marker 123. For example, the first marker can include two radiopaque bands 123a, 123b near the distal end of the sheath 110 that are separated a distance from one another so as to be identifiable under fluoroscopy (see FIGS. 10A-10C). The second marker can include a single radiopaque band 123c that is located a distance proximal to the first marker bands 123a, 123b. The single radiopaque band 123c can be positioned near the exit port 151 from the third lumen 150 to aid in positioning the port at a proper position for deployment of a device through the lumen 150. The second marker 123b can be positioned just proximal to the exit port 151, just distal to the exit port 151, or along a circumference incorporating the exit port 151. The markers 123a, 123b, 123c need not be in the shape of a solid band encircling the full circumference of the sheath 110. In some implementations, the distal markers 123a, 123b can incorporate two radiopaque bands whereas the exit port marker 123c can be a distinct mark at the location of the exit port 151, for example, encircling the port 151 itself as opposed to the entire circumference of the sheath 110 at that location.

The sheath 110 can incorporate a plurality of distal orientation markings 125 as discussed above with respect to FIGS. 1A-8D to signify circumferential alignment of the distal end region of the sheath 110 under fluoroscopy.

A proximal end region of the sheath 110 that is intended to remain outside the patient during use, such as the proximal hub 124, can additionally incorporate one or more visible markings 127 (see FIG. 9A). The proximal markings 127 need not be radiopaque as they are arranged on a region of the sheath 110 that is external to the patient so they are visible to the naked eye during a procedure. The proximal markings 127 can be applied according to any of a variety of known methods including an adhesive sticker, painted, molded, etched, embossed, or otherwise applied to a region of the sheath 110 external to the patient, such as the proximal hub 124, so that they are readily visible by a user during a procedure.

The proximal markings 127 can provide detectable guidance to a user with regard to the different lumens as well as to the circumferential orientation of the distal end region as discussed with regard to FIGS. 1A-8D. A proximal marking 127 can provide information on lumen size, distal opening location, and/or the intended purpose of the lumen (e.g., TAVR vs. embolic protection device) using different symbols, colors, and/or text. As an example, the lumen 150 having an exit port 151 positioned through the side wall can be identified with a marking 127 that indicates the side of the sheath 110 where the exit port 151 is located such as with an arrow pointing toward that side positioned next to the proximal opening 156 into that lumen 150. This can help to ensure the user will rotate the access sheath 110 in a manner so that the exit port 151 is properly positioned within the aortic arch, for example, so that an embolic protection filter inserted through the lumen 150 is deployed from a region of the catheter that is facing caudal or is located near the branch vessels off the arch. FIG. 10C shows the access sheath inserted via the left common carotid artery LCCA with a distal end region within the aortic arch between the descending aorta (DAo) and the Ascending aorta (AAo). The side exit is shown oriented towards the right carotid artery (RCCA). As another example, at least a region of the lumens including the proximal openings into the lumens can be colored a unique color to indicate a size of the lumen (e.g., green can indicate a largest lumen size for interventional device delivery, yellow a smaller lumen size for contrast or diagnostic catheter delivery, and red for the lumen having a side port). A proximal end region of lumen 120 and/or the proximal opening 126 into the lumen 120 can be a first color and a proximal end region and/or the proximal openings 136, 146 into the auxiliary lumens 130, 140 can be a second color that is different from the first color. A proximal end region of the lumen 150 and/or the proximal opening 156 into the lumen 150 can be a third color that is different from either the first and second color to indicate the lumen 150 is the one having the distal exit port 151 that is through a side wall of the distal end region of the sheath 110 and intended for embolic protection device deployment. The color can be combined with text or symbols. Any of a variety of combinations for the proximal markings 127 are considered herein to easily identify the various lumens of the sheath 110 in a manner that is easily identifiable by a user.

The hub 124 can incorporate a silicone slit valve or luer attachment to connect Touhy-Borst style. Each of the lumens 120, 130, 140, 150 can incorporate a dedicated hemostasis device. The sheath 110 can incorporate a flush port 113 and/or a flush port luer attachment to allow a user to flush all lumens simultaneously with saline, therapeutic agent, and/or contrast, or to record pressure. The hub 124 of the arterial access sheath 110 may include a Y-arm for delivery of contrast or saline flush, for aspiration, and/or may be fluidly connected to a shunt, wherein the shunt provides a shunt lumen or pathway for blood to flow from the arterial access sheath 110 to a return site such as a venous return site or a collection reservoir. In this regard, a retrograde or reverse blood flow state may be established in at least a portion of the artery. The Y-arm for inflation of an occlusion balloon on a distal end region of the sheath, if present, via an inflation lumen, and a hemostasis valve for introduction of an endovascular valve delivery system into the sheath.

Any of the sheaths described herein can have aspiration applied to the artery through it. In this regard, the access sheath 110 can be connected via a Y-arm to an aspiration source, so that embolic debris may be captured which may otherwise enter the remaining head and neck vessels, or travel downstream to lodge into peripheral vessels. The aspiration source may be active, for example a cardiotomy suction source, a pump, or a syringe. Alternately, a passive flow condition may be established, for example, by fluidly connecting the Y-arm to a shunt 112 (see FIG. 1), which in turn is connected to a lower-pressure source such as a collection reservoir at atmospheric or negative pressure, or a venous return site in the patient. The passive flow rate may be regulated, for example, by controlling the restriction of the flow path in the shunt.

Any of the sheaths 110 described herein can be constructed so that it can pass through or navigate bends in an artery without kinking. For example, if the access sheath 110 is being introduced into the common carotid artery (either left or right) in a retrograde fashion through the transcarotid approach, above the clavicle but below the carotid bifurcation, it is desirable that the elongated sheath body 115 be flexible while retaining hoop strength to resist kinking or buckling. This can be especially important in procedures that have limited amount of sheath insertion into the artery and/or where there is a steep angle of insertion as with a transcarotid access in a patient with a deep carotid artery and/or with a short neck. In these instances, there is a tendency for the sheath body tip to be directed towards the back wall of the artery due to the stiffness of the sheath. This causes a risk of injury from insertion of the sheath body itself, or from devices being inserted through the sheath into the arteries, such as guide wires. It is desirable to construct the sheath body 115 such that it can be flexed when inserted in the artery, while not kinking. The arterial access sheath 110 can be passed through bends of less than or equal to 45 degrees wherein the bends are located within 5 cm, 10 cm, or 15 cm of the arteriotomy measured through the artery. The sheath 110 may be generally straight with or without an angled bend at the tip of approximately 20°. The angled bend at the tip can aid in navigation and delivery.

The working portion of any of the arterial access sheaths 110 described herein, such as the sheath body 115 which enters the artery, can be constructed in two or more layers. An inner liner can be constructed from a low friction polymer such as PTFE (polytetrafluoroethylene), HDPE, or FEP (fluorinated ethylene propylene) to provide a smooth surface for the advancement of devices through the inner lumen. An outer jacket material can provide mechanical integrity to the inner liner and may be constructed from materials such as Pebax, thermoplastic polyurethane, polyethylene, nylon, or the like. The inner and outer surfaces can be lubricious through the selection of materials (PTFE, HDPE), coatings (silicone, hydrophilic coatings), and/or surface modifications. A third layer can be incorporated that can provide reinforcement between the inner liner and the outer jacket. The reinforcement layer can prevent flattening or kinking of the inner lumen of the sheath body as the device navigates through bends in the vasculature. The reinforcement layer can also provide for unimpeded lumens for device access as well as aspiration or reverse flow. In an embodiment, the sheath body 115 is circumferentially reinforced. The reinforcement layer can be made from metal such as stainless steel, Nitinol, Nitinol braid, helical ribbon, helical wire, cut stainless steel, or the like, or stiff polymer such as PEEK. The reinforcement layer can be a structure such as a coil and/or braid, or tubing that has been laser-cut or machine-cut so as to be flexible. In another embodiment, the reinforcement layer can be a cut hypotube such as a Nitinol hypotube or cut rigid polymer, or the like. The reinforcement of the elongate body 115 can be constructed so as to increase the longitudinal and torsional stiffness so that the sheath 110 has as near 1:1 movement from proximal hub 124 to distal tip 122.

Sheath retention may be achieved, for example, by an eyelet or other feature on a proximal end region of the access sheath 110 that allows the sheath to be secured to the patient once positioned correctly.

Initial access can be achieved using a micropuncture kit including an access needle, access guidewire, and micropuncture cannula. The access needle, access guidewire, and micropuncture cannula can be adapted to be introduced via a carotid puncture into the carotid artery or another vessel as discussed elsewhere herein. The carotid puncture may be accomplished, for example, percutaneously or via a surgical cut down. Upon establishment of access to the vessel, the access sheath 110 may then be inserted. The sheath guide wire can be inserted using a modified Seldinger technique or a micropuncture technique.

The access sheath 110 can be inserted with the help of a smooth tip sheath dilator configured for initial insertion over a sheath guide wire (e.g., 014, 018, 035). The sheath distal tip 122 can be configured such that when the access sheath 110 is assembled with the sheath dilator to form a sheath assembly, the sheath assembly can be inserted smoothly over the sheath guide wire through the arterial puncture with minimal resistance. Lubricious or hydrophilic coatings on at least a region of the sheath 110 can reduce friction during inserting into the vessel. The distal coating can be limited to a distal end region of the elongate body 115 so that the coating facilitates insertion without compromising security of the sheath in the puncture site or the ability of the operator to firmly grasp the sheath during insertion.

The elongate body 115 can vary in flexibility over its length. For example, the outer jacket may change in durometer and/or material at various sections. Alternately, the reinforcement structure or the materials may change over the length of the sheath body. In one embodiment, there is a distal-most section of sheath body 115, which is more flexible than the remainder of the sheath body. For example, the flexural stiffness of the distal-most section is one third to one tenth the flexural stiffness of the remainder of the sheath body 115. For a sheath configured for a CCA access site, the flexible, distal most section comprises a significant portion of the sheath body 115 may be expressed as a ratio. In an embodiment, the ratio of length of the flexible, distal-most section to the overall length of the sheath body 115 is at least one tenth and at most one half the length of the entire sheath body 115.

The access sheath and delivery system features described herein are useful for TAVR as well as a variety of other interventional methodologies, for example, in the treatment of coronary arteries, renal arteries, hepatic arteries, thoracic aortic aneurysms, and abdominal aortic aneurysms. Although the methods described herein are described in the context of TAVR, it should be appreciated that the sheaths described herein can be used for any of a variety of methods. The sheath 110 can be used to deploy any of a variety of devices through the first lumen 120 including prosthetic valves, differentially porous stents having asymmetrical braiding or coils that create areas of lesser or greater blood flow, fenestrated or branched devices, flow diverters configured to divert blood flow away from an aneurysm, fistula, or ruptured vessel may have a region that allows for flow to healthy tissue, vascular occluding devices that incorporate asymmetrical braids or differential lattice densities, and others. The sheath 110 can be used to deliver one or more of fluids (e.g., contrast agent, therapeutics, etc), devices including diagnostic catheters, pigtail catheters, electrophysiology pacing leads, pressure measurement devices, small bore catheters, microcatheters, and others, and to perform aspiration through one or more auxiliary lumens 130, 140. The sheath 110 can be used to deploy an embolic protection device through lumen 150 having a side port 151.

In an implementation, the access sheath 110 having multiple internal lumens is first inserted into the vasculature such as via either a percutaneous puncture or direct surgical cut down and puncture of the carotid artery. The access sheath 110 can be introduced through the penetration with the tip directly inferiorly towards the ostium of the aorta and, optionally, oriented such that lumen 150 is directed towards the embolic protection device target. A transcarotid approach to the aortic valve may be achieved via the LCCA or via the RCCA. FIG. 10C illustrates an approach through the LCCA. Once the access sheath is positioned and optionally the embolic protection means are deployed through lumen 150 via occlusion, aspiration, and/or filter elements, access to the aortic valve is obtained via a guidewire (such as a 0.035″ or 0.038″ guidewire) inserted into the sheath 110 through first lumen 120 and directed inferiorly into the ascending aorta and across the native aortic valve or previously implanted bio-prothesis. A diagnostic catheter (e.g. pigtail catheter) may be inserted through lumen 130 and delivered superiorly to the aortic valve to enable direct contrast injection and visualization via fluoroscopy. Lumen 140 may be used at any time during the procedure to deliver additional ancillary devices, perform aspiration, and/or inject contrast superior to the aortic valve. Pre-dilation of the native aortic valve can be performed with an appropriately sized dilation balloon, for example a valvuloplasty balloon, before valve implantation. The guidewire is used to position a balloon across the valve and the balloon is inflated, deflated, and then removed while the guidewire remains in place. An endovascular prosthetic valve and delivery system is then inserted through the lumen 120 of the access sheath 110 over the guidewire. The valve positioned on the delivery system can be tracked through the access sheath 110 to the site of the native aortic valve. The prosthetic valve can be adjusted if needed longitudinally along the long axis and/or circumferentially around the long axis until the desired alignment is achieved. The prosthetic valve is then deployed at or near the position of the native aortic valve. The approach from the carotid artery can increase the circumferential control of the sheath 110 and the prosthetic to improve accuracy of alignment.

At the conclusion of the implantation step, the implanted prosthetic valve function can be accessed via ultrasound, contrast injection under fluoroscopy, or other imaging means. Depending on the design of the delivery system, the prosthetic valve may be adjusted as needed to achieve optimal valve function and position before final deployment. The delivery system and guidewire are then removed from the access sheath 110. The diagnostic catheter is then removed from the access sheath 110. After removal of the delivery system and guidewire, any embolic protection elements are removed from lumen 150. Aspiration may continue through lumen 140 during this time to capture any embolic debris caught in the sheath tip, occlusion element and/or filter elements. Various forms of embolic protection can be deployed including occlusion elements, filters, occlusion balloons, aspiration, and the like.

The access sheath 110 is then removed and the access site is closed. If the access was a surgical cutdown direct puncture, the vessel is closed either via tying off the pre-placed stitch or with manual suturing or with a surgical vascular closure device, as described in more detail below. If the access was percutaneous, percutaneous closure methods and devices may be employed to achieve hemostasis at the access site. In an implementation, the closure device is applied at the site of the penetration before introducing the arterial access sheath through the penetration. The type of closure device can vary.

The access site described above is either the left or right common carotid artery. Other access sites are also possible, for example the left or right subclavian artery or left or right brachial artery or transfemoral approach. These arteries may require longer and/or more tortuous pathways to the aortic valve but may offer other advantages over a carotid artery access, for example the ability to work away from the patient's head, the ability to avoid hostile neck anatomy such as previous carotid endarterectomy or other cervical surgery or radiation, or less risky in case of access site complication. In addition, carotid artery disease, or small carotid arteries may preclude common carotid artery access. In the case of any of these access sites, occlusion, aspiration, and/or filtering the head and neck vessels during TAVI may increase the speed and accuracy of the procedure, and decrease the rate of embolic complications.

If the access to the carotid artery for any of the methods described herein was via a surgical cut down, the access site may be closed using standard vascular surgical techniques. Purse string sutures may be applied prior to sheath insertion, and then used to tie off the access site after sheath removal. If the access site was a percutaneous access, a wide variety of vessel closure elements may be utilized. In an implementation, the vessel closure element is a mechanical element which include an anchor portion and a closing portion such as a self-closing portion. The anchor portion may comprise hooks, pins, staples, clips, tine, suture, or the like, which are engaged in the exterior surface of the common carotid artery about the penetration to immobilize the self-closing element when the penetration is fully open. The self-closing element may also include a spring-like or other self-closing portion which, upon removal of the sheath, will close the anchor portion in order to draw the tissue in the arterial wall together to provide closure. Usually, the closure will be sufficient so that no further measures need be taken to close or seal the penetration. Optionally, however, it may be desirable to provide for supplemental sealing of the self-closing element after the sheath is withdrawn. For example, the self-closing element and/or the tissue tract in the region of the element can be treated with hemostatic materials, such as bioabsorbable polymers, collagen plugs, glues, sealants, clotting factors, or other clot-promoting agents. Alternatively, the tissue or self-closing element could be sealed using other sealing protocols, such as electrocautery, suturing, clipping, stapling, or the like. In another method, the self-closing element will be a self-sealing membrane or gasket material which is attached to the outer wall of the vessel with clips, glue, bands, or other means. The self-sealing membrane may have an inner opening such as a slit or cross cut, which would be normally closed against blood pressure. Any of these self-closing elements could be designed to be placed in an open surgical procedure, or deployed percutaneously.

In an alternate implementation, the vessel closure element is a suture-based vessel closure device. The suture based vessel closure device can place one or more sutures across a vessel access site such that, when the suture ends are tied off after sheath removal, the stitch or stitches provide hemostasis to the access site. The sutures can be applied either prior to insertion of a procedural sheath through the arteriotomy or after removal of the sheath from the arteriotomy. The device can maintain temporary hemostasis of the arteriotomy after placement of sutures but before and during placement of a procedural sheath and can also maintain temporary hemostasis after withdrawal of the procedural sheath but before tying off the suture. U.S. Pat. No. 8,858,490, entitled “SYSTEMS AND METHODS FOR TREATING A CAROTID ARTERY”, which is incorporated herein by reference in its entirety, describes exemplary closure devices and also describes various other devices, systems, and methods that are related to and that may be combined with the devices, systems, and methods disclosed herein.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. An arterial access sheath for delivering a transcatheter aortic valve treatment, the sheath comprising:
an elongate body having at least one internal lumen extending from a proximal opening to a distal opening;
a proximal hub coupled to the proximal opening of the elongate body and configured to remain external to a patient, the proximal hub comprising one or more proximal orientation markings; and
a radiopaque marker band embedded within a distal end region of the elongate body, the radiopaque marker band comprising cut-outs separated from one another around a circumference of the distal end region corresponding circumferentially to the one or more proximal orientation markings,
wherein the cuts-outs form gaps in radiopacity in the marker band creating at least two non-radiopaque distal orientation markings,
wherein overlap of the at least two distal orientation markings indicates circumferential orientation of the distal end region of the elongate body.

2. The arterial access sheath of claim 1, wherein the elongate body has a length adapted to be introduced into an access site at a left or a right common carotid artery, or a left or a right subclavian artery.

3. The arterial access sheath of claim 1, wherein a shape of the distal orientation markings forms a second, different shape upon at least partial overlap of the at least two distal orientation markings with one another.

4. The arterial access sheath of claim 3, wherein the shape is a rectangle, circle, triangle, diamond, arrow, hemisphere, or square.

5. The arterial access sheath of claim 1, wherein the at least two distal orientation markings comprises at least two groups of distal orientation markings.

6. The arterial access sheath of claim 5, wherein the at least two groups are distinguishable from each other.

7. The arterial access sheath of claim 6, wherein the at least two groups are distinguishable by a shape of markers.

8. The arterial access sheath of claim 5, wherein the at least two groups are distinguishable by a number of markers.

9. The arterial access sheath of claim 1, wherein the radiopaque marker band comprises a polymer loaded with one or more radiopaque materials.

10. The arterial access sheath of claim 9, wherein the radiopaque materials is selected from the group consisting of tungsten, tantalum, barium sulfate, platinum, stainless steel, and gold.

11. The arterial access sheath of claim 1, wherein the distal orientation markings additionally include a button of radiopaque material projecting away from a sidewall of the distal end region of the elongate body.

12. The arterial access sheath of claim 1, wherein the proximal orientation markings are on a proximal-facing surface of the hub and are directly visible by a user.

13. The arterial access sheath of claim 1, wherein the proximal orientation markings comprise adhesive markings, painted markings, molded markings, etched markings, embossed markings, or printed marking on the proximal hub.

14. The arterial access sheath of claim 1, wherein the elongate body comprises an asymmetry around its circumference, wherein the proximal orientation markings correspond to the asymmetry.

15. The arterial access sheath of claim 1, wherein the elongate body is non-circular in cross-section.

16. A method of treating an aortic valve, comprising:
forming a penetration through the neck of a patient in a wall of a common carotid artery;
introducing an arterial access sheath through the penetration, the access sheath comprising an elongate body coupled to a proximal hub, the proximal hub configured to remain external to a patient and comprising one or more proximal orientation markings, wherein a distal end region of the elongate body comprises a radiopaque marker band comprising cut-outs separated from one another around a circumference of the distal end region so as to correspond circumferentially to the one or more proximal orientation markings, wherein the cut-outs form gaps in radiopacity in the marker band creating at least two non-radiopaque distal orientation markings;
aligning the distal end region of the elongate body relative to a target anatomy under fluoroscopy according to an overlapping circumferential position of the at least two distal orientation markings;
introducing a prosthetic valve through the access sheath using as a guide the one or more proximal orientation markings corresponding to the at least two distal orientation markings; and
deploying the prosthetic valve at or near the position of a native aortic valve.

17. An arterial access sheath for delivering a transcatheter aortic valve treatment, the sheath comprising:
an elongate body comprising:
a plurality of internal lumens extending parallel to one another between respective proximal openings and respective distal openings, the distal openings located at a distal-most end of the elongate body;
an embolic protection device lumen extending between a proximal opening and an exit port, the exit port located through a sidewall of the elongate body a distance proximal to the distal-most end of the elongate body;
a first radiopaque marker band embedded within a distal end region of the elongate body; and
a second radiopaque marker band embedded within a region of the elongate body near the exit port; and
a proximal hub configured to remain external to a patient, the proximal hub coupled to the respective proximal openings of the elongate body and the proximal opening of the embolic protection device lumen.

18. The arterial access sheath of claim 17, wherein the plurality of internal lumens comprises an interventional device lumen having an inner diameter that is between 12 French and 24 French or between 4 mm and 8 mm.

19. The arterial access sheath of claim 18, wherein the plurality of internal lumens comprises at least two auxiliary lumens having an inner diameter that is between 3 French and 9 French or between 1 mm and 3 mm.

20. The arterial access sheath of claim 17, further comprising a flush valve on the proximal hub configured to simultaneously flush the plurality of internal lumens.

21. The arterial access sheath of claim 17, wherein the distance is at least about 10 mm up to about 30 mm.

22. The arterial access sheath of claim 17, wherein the elongate body has a length adapted to be introduced into an access site at a left or right common carotid artery, or a left or right subclavian artery such that the distal-most end is positionable within the descending aorta.

23. The arterial access sheath of claim 22, wherein the length is between about 10 cm up to about 50 cm.

24. The arterial access sheath of claim 17, wherein the proximal hub comprises one or more proximal orientation markings that are directly visible by a user.

25. The arterial access sheath of claim 24, wherein the proximal orientation markings are on a proximal-facing surface of the hub.

26. The arterial access sheath of claim 24, wherein at least one of the first and second radiopaque marker bands comprises cut-outs separated from one another around a circumference of the distal end region so as to correspond circumferentially to the one or more proximal orientation markings, wherein the cut-outs form gaps in radiopacity in the marker band creating at least two non-radiopaque distal orientation markings.

27. The arterial access sheath of claim 26, wherein overlap of the at least two distal orientation markings indicate circumferential orientation of the distal end region of the elongate body.

28. A method of treating a vessel using an arterial access sheath comprising an elongate body having a plurality of internal lumens and a proximal hub, the method comprising:
inserting the elongate body through a penetration in a wall of a vessel of a patient so that the proximal hub is accessible from outside the patient;
inserting a first device into a first proximal opening into a first lumen of the plurality of internal lumens through the proximal hub and out a distal opening from the first lumen; and
inserting a second device into a second proximal opening into a second lumen of the plurality of internal lumens through the proximal hub and out an exit port from the second lumen,
wherein the first lumen and the second lumen have different internal diameters, and wherein the distal opening from the first lumen is located at a distal-most end of the elongate body and wherein the exit port from the second lumen is located through a sidewall of the elongate body a distance proximal to the distal-most end.

29. The method of claim 28, wherein the elongate body further comprises at least a first radiopaque marker embedded within a distal end region of the elongate body near the distal opening and a second radiopaque marker embedded within a region of the elongate body near the exit port.

30. The method of claim 29, wherein the region of the elongate body having the second radiopaque marker is located adjacent a proximal side of the exit port, adjacent a distal side of the exit port, or along a circumference incorporating the exit port.

31. The method of claim 28, wherein the first device comprises an endovascular valve delivery system and the second device comprises an embolic protection device.

32. The method of claim 28, wherein the proximal hub comprises a plurality of proximal markers providing information about the plurality of internal lumens.

33. The method of claim 32, wherein the information comprises at least one of lumen size, distal opening location, and purpose of lumen.
